(19) United States
(12) Patent Application Publication
(10) Pub. No.: US 2024/0366283 A1
(43) Pub. Date: Nov. 7, 2024

(54) ELECTROSURGICAL SYSTEM AND METHOD FOR THE OPERATION THEREOF

(71) Applicant: Erbe Elektromedizin GmbH, Tuebingen (DE)

(72) Inventors: Christof DIERL, Bondorf (DE); Thomas FRASCH, Kusterdingen (DE); Michael SAUTER, Albstadt (DE); Marc KEGREISS, Rottenburg (DE)

(21) Appl. No.: 18/652,895

(22) Filed: May 2, 2024

(30) Foreign Application Priority Data
May 5, 2023 (EP) 23171927.9

(51) Int. Cl. A61B 18/12; A61B 18/00; A61B 18/16

(52) U.S. Cl. CPC A61B 18/1206; A61B 18/16; A61B 2018/00648; A61B 2018/00702; A61B 2018/0072; A61B 2018/00732; A61B 2018/00761; A61B 2018/00767; A61B 2018/00779; A61B 2018/00827; A61B 2018/00875; A61B 2018/00886; A61B 2018/00892; A61B 2018/00904; A61B 2018/1253; A61B 2018/126

(57) ABSTRACT

An electrosurgical system and a method for the operation thereof are disclosed. The electrosurgical system includes a supply apparatus and an electrosurgical instrument connected thereto having at least one instrument electrode. By means of the instrument electrode, biological tissue of a patient can be treated, for example by creation of a spark between the instrument electrode and tissue. The supply apparatus evaluates at least one operating parameter of the operating circuit, which includes the electrosurgical instrument and the treated tissue, and which is supplied with electrical power by the supply apparatus. A tissue type of the treated tissue is recognized by evaluation of at least one operating parameter. Thereupon at least one electrical parameter of the provided electrical power can be adapted or modified so that the electrical power provided to the electrosurgical instrument is optimized for the tissue type of the currently treated tissue.

### CROSS REFERENCE TO RELATED APPLICATION

This application claims the benefit of European Patent Application No. 23171927.9, filed May 5, 2023, which is incorporated herein by reference in its entirety.

### TECHNICAL FIELD

The invention refers to an electrosurgical system and a method for the operation thereof. The electrosurgical system comprises a supply apparatus as well as an electrosurgical instrument having at least one instrument electrode. The electrosurgical instrument is configured for treatment of biological tissue. During treatment it is connected to the supply apparatus of the electrosurgical system. When using the electrosurgical instrument an operating circuit is closed or formed, which leads from an electrical connection of the supply apparatus via an instrument electrode of the electrosurgical instrument, the tissue to be treated and another electrode-optionally a neutral electrode, which is separate from the instrument and which is attached to the patient-back to a connection of the supply apparatus. The neutral electrode is present if the electrosurgical instrument is a monopolar instrument. In case of a bipolar instrument having multiple instrument electrodes, a separate neutral electrode can be omitted.

The supply apparatus of the electrosurgical instrument provides an electrical power in form of an electrical operating voltage and/or an electrical operating current for the electrosurgical instrument. Optionally also additional operating media can be provided, for example an operating fluid.

### BACKGROUND

Electrosurgical systems of this type and their operation during treatment of biological tissue of a patient are known from the prior art. Different tissue types require different operating settings in order to provide a treatment adapted to the respective tissue type. An operating person, particularly a surgeon, can therefore select or adjust a desired operating mode on the electrosurgical system (for example on the supply apparatus), which is also denoted as mode. This requires expert knowledge of the operating person in order to adjust an optimum mode or to select an optimum mode from the available modes for the respectively intended treatment of the tissue.

In order to simplify the use of the electrosurgical system for the operating person, it is known from the prior art to carry out an automatic recognition of the treated tissue type and to optionally automatically switch from one mode to another mode. Such a method for operating an electrosurgical system is described in DE 10 2020 105 835 A1, for example. There it is proposed for determination of the tissue type to carry out an optical tissue type recognition, because a tissue type recognition exclusively based on electrical characteristics of the tissue would be only possible in a limited manner.

EP 3 876 238 A1 discloses a method and a system for support of surgical operations. During the treatment measurement data are determined and temporarily stored in a supply apparatus. The temporally stored measurement data are then transmitted to a central evaluation device. In the central evaluation device the received data are stored and evaluated. Based on this evaluation, available modes of the supply apparatus can be modified, completely cancelled or new modes can be created.

### SUMMARY

The modes provided by a supply apparatus of an electrosurgical system serve different treatment methods of biological tissue and can therefore remarkably distinguish from one another. For this reason, the automatic tissue type recognition has to be carried out with sufficient reliability and very quickly, for example if the tissue type changes only temporarily during treatment. For example, this can occur if a treated tissue type is interfused with smaller locally limited structures of another tissue type.

It can be considered as one object of the present invention to provide an electrosurgical system and a method for the operation thereof that allows to quickly and reliably adapt the operation to different tissue types.

This object is solved by an electrosurgical system as well as a method for operation of an electrosurgical system as described herein.

The electrosurgical system according to the invention comprises a supply apparatus as well as an electrosurgical instrument connected or connectable to the supply apparatus. The electrosurgical instrument has at least one instrument electrode by means of which a patient's biological tissue to be treated can be influenced. Thereby the at least one instrument electrode can be arranged with distance to the tissue or can be in contact with the tissue. During use an electrical spark can form between the instrument electrode and the tissue.

The electrosurgical instrument is particularly configured for cutting and/or coagulating of the tissue to be treated. It can be provided and configured to be used in open surgical or endoscopic manner. The at least one instrument electrode can have a geometric form and dimension suitable for the respective treatment, whereby it can be a needle-shaped instrument electrode or a spatula-shaped instrument electrode, for example.

The electrosurgical instrument can be a monopolar instrument. In order to close the operating circuit, in this case a separate electrode that can be denoted as neutral electrode is electrically conductively arranged on the patient. An operating current can then flow from the supply apparatus via the at least one instrument electrode of the electrosurgical system, the treated tissue and the neutral electrode back to the supply apparatus. In this manner a closed operating circuit is formed. If the instrument is a bipolar instrument, the operating current can be closed by the multiple instrument electrodes present on the electrosurgical instrument, so that a separate neutral electrode can be omitted.

The supply apparatus is configured to provide an operating voltage and/or an operating current for the operating circuit or the electrosurgical instrument. Thereby either the operating voltage or the operating current can be preset or impressed by the supply apparatus (for example a controllable generator of the supply apparatus). The settings for the operating voltage and/or the operating current thereby depend on the selected or set mode of the supply apparatus.

Each mode is characterized by a mode parameter set having multiple mode parameters. One or more of the following parameters can be used as mode parameter for forming a mode parameter set: a maximum value and/or an average value and/or a root mean square value of the operating voltage; a maximum value and/or an average value and/or a root mean square value of the operating current; a modulation type for the operating voltage and/or for the operating current; a crest factor of the operating voltage and/or the operating current and/or a parameter correlated therewith such as an electrical operating power; a frequency of the operating voltage and/or the operating current; a waveform of the operating voltage and/or the operating current, for example a square wave, sinusoidal or another waveform, which can be preset continuously (also denoted as “continuous wave” or “cw”) or quasi-continuously having short pulse durations and/or pulse pauses; a direct voltage value of the operating voltage or a direct current value of the operating current, wherein the direct voltage or direct current can be applied continuously or can be switched on and off according to a preset time interval.

A quasi-continuously waveform means particularly a waveform that provides an alternating signal, particularly high frequency alternating signal for the operating voltage and/or the operating current, which is repeatedly continuously applied during a pulse duration (in form of a pulse or burst so-to-speak), wherein between subsequent pulses or pulse durations a pulse pause is present. During the pulse pause the operating voltage and/or the operating current is equal to zero or at least negligibly small, so that tissue is not influenced. The pulse duration and the pulse pause have preferably equal durations, however, can also have different durations. The pulse duration and/or the pause duration can, for example, have an amount of maximum 10 ms or maximum 5 ms.

According to the invention, the supply apparatus is configured to detect at least one electrical operating parameter in the operating circuit resulting from the operation of the electrosurgical system. The at least one operating parameter can be an operating parameter, which is preset by the supply apparatus and/or an operating parameter which results from the use depending on the treated tissue and optionally additional external conditions. Particularly, at least the operating voltage and/or the operating current are measured as operating parameters. As an option, depending on the operating voltage and/or the operating current, in addition at least one additional operating parameter can be calculated, estimated or determined in another manner. For example, at least one of the following parameters can be used as operating parameter: an ohmic resistance and/or the impedance of the operating circuit; a spark parameter that characterizes an influence of a spark between the instrument electrode and the treated tissue on the operating current and/or the operating voltage; an active power; a reactive power; an apparent power; a power factor; an arbitrary voltage value resulting from the determination of the operating voltage, for example a maximum value and/or an average value and/or a root mean square value; an arbitrary current value resulting from the measurement of the operating current, particularly a maximum value and/or an average value and/or a root mean square value of the operating current.

Based on the at least one detected operating parameter it is checked whether a tissue type of the tissue treated by means of the electrosurgical system changes from a primary tissue type, which shall be treated by means of the set mode, to a secondary tissue type, which is different to the primary tissue type. For example, the tissue type can change from connective tissue (primary tissue type) to the tissue of a blood vessel (secondary tissue type). If such a change is determined, the supply apparatus adapts at least one mode parameter of the mode parameter set at least temporarily, in order to consider the change of the tissue type from the primary tissue type to the secondary tissue type. For example, the mode parameter to be adapted is thereby changed from a respectively assigned primary parameter value or primary condition to a secondary parameter value or secondary condition different from the primary parameter value or primary condition. For example, for this purpose a voltage value of the operating voltage and/or a current value of the operating current and/or a modulation type of the operating voltage or the operating current and/or a waveform of the operating voltage or the operating current can be modified.

The monitoring of the at least one operating parameter of the operating circuit by means of the supply apparatus is possible very quickly and with very simple means, particularly in time intervals in the millisecond range, particularly maximum 100 ms or maximum 50 ms. In doing so, an adaption of the at least one mode parameter can be carried out very quickly in order to be able to react sufficiently quickly to very small tissue structures of the secondary tissue type. For example, by means of the invention an adaption can be carried out very quickly if the set mode for treating and particularly for cutting of connective tissue is provided and the connective tissue is interfused locally by small blood vessels, the tissue type of which (secondary tissue type) differs from the primary tissue type of the connective tissue.

This adaption of the at least one mode parameter is particularly carried out without leaving the set mode and without switching to another selectable mode.

Preferably each mode parameter within one respective mode parameter set comprises a defined value range. It is particularly provided that the mode parameter does not leave this value range. During adaption of the at least one mode parameter within the set mode, the defined value range is therefore not left.

It is in addition advantageous if the supply apparatus is configured to change an adapted mode parameter again back to its initial setting if it is determined that the tissue type changes once again from the secondary tissue type to the primary tissue type.

Additionally or alternatively, a termination condition can be defined upon fulfillment of which the supply apparatus changes the at least one mode parameter again to its initial setting of the respective mode parameter. Such a termination condition can be fulfilled, for example if a predefined maximum time duration has expired since the one or more mode parameter was changed. The maximum time duration can be, for example, 5 seconds or 2 seconds or 1 second. This optional measure can provide an additional safety during treatment of biological tissue. The mode set or selected by the operating person is thereby only modified for a short period if the secondary tissue type has been recognized. This automatic adaption or modification of at least one mode parameter can be temporarily limited in order to leave the decision-making power over the set mode parameter to the operating person. The fulfillment of the termination condition can be indicated to the operating person. If over a longer period another tissue type than the primary tissue type shall be treated, a modified mode setting can be potentially advantageous or necessary.

As already explained, the electrosurgical system is in one embodiment configured to recognize connective tissue as one tissue type and a blood vessel as another tissue type and/or at least to distinguish them from one another. Preferably, the connective tissue is the primary tissue type and the tissue of the blood vessel is the secondary tissue type.

For recognition of the tissue type, particularly connective tissue and/or blood vessel, or for recognition of the change of the tissue type, particularly from connective tissue to a blood vessel or vice versa from a blood vessel to a connective tissue, preferably a combination of multiple operating parameters can be considered:
the resistance or impedance of the operating circuit;
a change of the resistance or the impedance of the operating circuit exceeding an upper threshold or dropping below a lower threshold;
the spark parameter;
a change of a spark parameter within a predefined change duration about a minimum value;
the spark parameter exceeds or drops below a predefined threshold;
the power factor of the electrical power of the operating circuit changes about a minimum absolute value within a predefined change duration.

For example, a blood vessel can be recognized if
(a) the ohmic resistance of the operating circuit is less than 500 Ohm (preferably remarkably smaller),
(b) the spark parameter exceeds a predefined threshold within a change duration of preferably maximum 5 ms and
(c) the power factor is within a predefined tolerance range around the absolute value of 20%, for example within a tolerance range from 15% to 25%.

Preferably connective tissue can be recognized in that
(a) the resistance of the operating circuit is above a threshold of 1500 Ohm,
(b) the spark parameter is below a predefined threshold during a minimum duration, for example 10 ms, and
(c) the power factor increases or decreases within a preset change duration, for example 50 ms, remarkably between 1% and 100%, for example about a percent value of at least 50% or 60% or 70%.

One mode parameter or multiple of the following mode parameters can be used as mode parameter:
maximum value and/or average value and/or root mean square value of the operating voltage,
maximum value and/or average value and/or root mean square value of the operating current,
active power and/or apparent power and/or reactive power provided to the operating circuit,
a power factor, particularly indicating the ratio of the active power to the apparent power,
a frequency form and/or a waveform of the operating voltage,
a frequency form and/or a waveform of the operating current,
a modulation type of the operating voltage and/or the operating current.

If according to the invention by means of the evaluation of the at least one operating parameter the transition from connective tissue as primary tissue type to a blood vessel as secondary tissue type has been determined, the mode parameter set can be adapted as follows:
the allowable maximum absolute value of the operating voltage is reduced from a primary voltage value to a secondary voltage value; and/or
the allowable maximum absolute value of the operating current is increased from a primary current value to a secondary current value; and/or
the modulation type of the currently used waveform (particularly high frequency alternating signal form) for the operating voltage and/or the operating current is changed to a quasi-continuous waveform for the operating voltage and/or the operating current.

The high frequency alternating signal is particularly preset so that a polarity change of the operating voltage or the operating current occurs. The operating voltage and/or the operating current can have a sinusoidal progress.

In an embodiment the adaption of the at least one mode parameter is indicated, for example, optically and/or acoustically and/or haptically. For this purpose, an operating interface on the supply apparatus can be used, for example. Additionally or alternatively, the indication can also provided on the electrosurgical instrument, for example by means of a haptic signal, such as a vibration signal. Each automatic change of a mode parameter or of multiple mode parameters of a mode parameter set can be indicated to an operating person.

During treatment of primary tissue type with the set mode at least one or exactly one mode parameter can be dynamically adapted to the tissue impedance of the treated tissue (primary tissue type). For example, the at least one automatically adaptable mode parameter can comprise:
the crest factor of the operating voltage and/or
the crest factor of the operating current and/or
the crest factor of another electrical parameter depending on the operating voltage and/or the operating current such as the electrical operating power.

The adaption range, within which the respective mode parameter can be adapted automatically, can optionally be limited by an upper limit value and/or a lower limit value. Additionally or alternatively, also at least one additional electrical parameter can be limited, which may change as a result of the automatic adaption of the at least one mode parameter, by an assigned upper limit value and/or lower limit value.

Any embodiment of the electrosurgical system described herein is operated as follows according to the invention:

First a mode is set, which is defined by a mode parameter set of multiple mode parameters. Based on this mode, the operating circuit is provided with an electrical operating voltage and/or an electrical operating current by the supply apparatus. During the operation and particularly during treatment of biological tissue, at least one electrical operating parameter of the operating circuit is detected, particularly the operating voltage and the operating current are continuously measured or determined in another manner. Based on the operating voltage and/or the operating current, one or more additional operating parameters can be determined.

Based on the at least one operating parameter it is evaluated whether the treated tissue type changes from a primary tissue type assigned to the set mode to a secondary tissue type different therefrom. In this case, at least one mode parameter of the mode parameter set of the set mode is adapted at least temporarily to the secondary tissue type.

### BRIEF DESCRIPTION OF THE DRAWINGS

Advantageous embodiments of the invention are derived from the dependent claims, the description and the drawing. In the following preferred embodiments of the invention are explained in detail with reference to the attached drawing. The drawing shows:

FIG. 1 a schematic block-diagram-like illustration of an embodiment of an electrosurgical system,

FIG. 2 a schematic illustration of available modes for operating the electrosurgical system of FIG. 1, wherein each mode comprises a mode parameter set having multiple mode parameters,

FIG. 3 a principle illustration of using an electrosurgical instrument of the electrosurgical system of FIG. 1 during treatment of biological tissue,

FIGS. 4 and 5 highly schematic qualitative illustrations of exemplary temporary changes of operating parameters of the electrosurgical system of FIG. 1 and

FIG. 6 highly schematic exemplary temporal progresses of multiple mode parameters during a determined change of a tissue type treated by means of the electrosurgical instrument.

### DETAILED DESCRIPTION

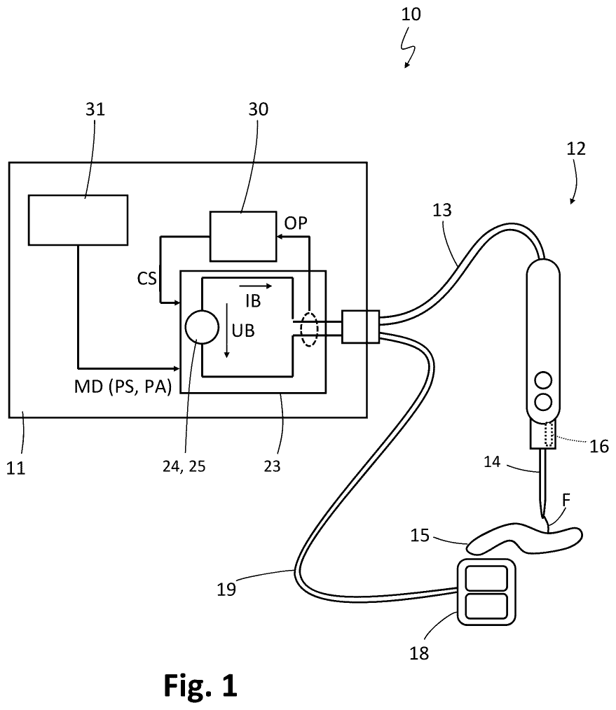

In FIG. 1 an embodiment of an electrosurgical system 10 is illustrated schematically in the type of a block diagram. The electrosurgical system 10 comprises a supply apparatus 11 as well as an electrosurgical instrument 12 connected or connectable to the supply apparatus 11. A line or cable 13 having at least one electrical conductor serves for electrical connection of the electrosurgical instrument 12 to the supply apparatus 11. The electrosurgical instrument 12 has at least one instrument electrode 14 by means of which a patient's biological tissue to be treated can be influenced.

In addition to the electrical connection via cable 13, also an optical and/or fluidic connection can be established between supply apparatus 11 and electrosurgical instrument 12, wherein in this case the cable 13 comprises respective optical and/or fluidic lines. For example, an optical detection device 16 can be provided on the electrosurgical instrument 12 in order to detect light that can be created between instrument electrode 14 and the tissue 15 during influence on tissue 15, for example if a spark F is formed between instrument electrode 14 and tissue 15, as schematically illustrated in FIGS. 1 and 3. The optical detection device 16 can be connected with supply apparatus 11 by means of an optical conductor of cable 13 in order to supply the received light to the supply apparatus 11.

In the embodiment illustrated in FIG. 1 the electrosurgical instrument 12 is configured as monopolar instrument. One single instrument electrode 14 is, therefore, sufficient. To form a closed operating circuit 17 with the monopolar instrument, in addition to the instrument electrode 14 a separate electrode is present that is or can be electrically conductively attached on the patient. This separate electrode can be denoted as neutral electrode 18. The neutral electrode 18 is electrically conductively connected to the supply apparatus 11 by means of an electrical line 19.

In modification to the illustrated embodiment, the electrosurgical instrument 12 can also be a bipolar instrument having at least two instrument electrodes 14 that can have different electrical potentials. The operating circuit 17 can then be closed via one of the instrument electrodes 14, the tissue 15 and another instrument electrode of the bipolar instrument 12. A separate neutral electrode 18 can be omitted in this case.

The electrosurgical instrument 12 is illustrated in the drawing as surgical instrument for the open surgical use. Alternatively to this, it can also be configured for the endoscopic use and in this case have a configuration so that the electrosurgical instrument 12 can be inserted through the endoscope channel of an endoscope into the patient.

Depending on the application, the at least one instrument electrode 14 can have different configurations. For example, it can be needle-shaped or spatula-shaped. The at least one instrument electrode 14 can be immovably fixated on a handle of the electrosurgical instrument 12 or alternatively can be supported in a forceps or scissors-like manner, particularly in case of a bipolar instrument having multiple instrument electrodes 14.

The supply apparatus 11 is configured to supply electrical energy or electrical power to the electrosurgical instrument 12 by means of which the at least one instrument electrode 14 can treat biological tissue 15. The electrosurgical instrument 12 can be configured to cut and/or coagulate tissue 15, for example. For this purpose, the supply apparatus 11 comprises in the embodiment a generator 23, which can comprise a voltage source 24 and/or a current source 25. By means of generator 23 an operating voltage UB and/or an operating current IB can be provided at an output 26 of supply apparatus 11. Depending on the configuration of the generator 23, either the operating voltage UB or the operating current IB can be preset or impressed. The respective other parameter that is not preset or not impressed (operating current IB or operating voltage UB) then results depending on the electrical characteristics of the operating circuit 17 connected to the output 26, particularly on its electrical resistance R or its impedance.

During use of the electrosurgical system 10 the operating circuit 17 is closed so that an operating current IB can flow. In case of a monopolar instrument, it leads from a connection of the supply apparatus 11 (or generator 23) via an electrical conductor and in case of the monopolar instrument cable 13, the instrument electrode 14, the treated tissue 15, the neutral electrode 18 and the electrical line 19 back to another connection of supply apparatus 11 (or generator 23). In case of a bipolar instrument, the operating circuit 17 is closed by another instrument electrode 14 and another electrical conductor of cable 13 instead of the neutral electrode 18.

In any case the treated tissue 15 is part of the operating circuit 17 and the characteristic of the tissue 15 influences the electrical characteristics of the operating circuit 17, for example the ohmic resistance R or the impedance of the operating circuit 17.

According to the example, supply apparatus 11 comprises an evaluation unit 30. The evaluation unit 30 is configured to measure and/or calculate and/or estimate at least one operating parameter OP of operating circuit 17 or to determine it in another suitable manner. In the embodiment by means of the evaluation unit 30 the operating voltage UB and/or the operating current IB are measured continuously or in regular time intervals as operating parameter OP. Thereby the operating voltage UB as well as the operating current IB can be measured. Alternatively, it is also possible that the parameter impressed by generator 23 is not measured, because it is known due to the setting of the generator 23 in the supply apparatus 11 and the preset, impressed desired value is used as actual value.

The evaluation unit 30 can additionally or alternatively determine at least one operating parameter OP from one or more of the measured operating parameters OP. As operating parameter OP one or more of the following parameters can be used:
an ohmic resistance R and/or an impedance and/or the absolute value of the impedance of the operating circuit 17,
a maximum value and/or an average value and/or a root mean square value and/or a crest factor of the operating voltage UB and/or the operating current IB,
an active power and/or reactive power and/or apparent power provided to the operating circuit 17,
a power factor, for example indicating the ratio of the active power to the apparent power,
a spark parameter FP describing the influence of a spark F between the instrument electrode 14 of electrosurgical instrument 12 and the treated tissue 15 on the operating current IB and/or on the operating voltage UB.

The spark parameter FP can be derived from a distortion of the measured operating current IB and/or the measured operating voltage UB. For example, it can describe or can be determined based on a non-linear component of the electrical parameter (operating current IB or operating voltage UB), which is respectively not preset or impressed by generator 23. This non-linear component mainly results from the non-linear resistance created by spark F. For this purpose, the evaluation unit 30 can be configured to determine the correlation between the operating current IB and the operating voltage UB based on a linear equivalent circuit diagram and to compare it with the measured operating current IB and/or the measured operating voltage UB. A difference occurring thereby characterizes the non-linearity of spark F in the operating circuit 17 and can serve as basis for determination of the spark parameter.

For example, the determined difference can be related to the respectively assigned measured parameter and thereby the spark parameter FP can be determined. For example, if the operating voltage UB is impressed, the operating current IB can be measured and the difference from the calculated value for the operating current IB and the measured value for the operating current IB can be calculated. The root mean square value of this difference and a root mean square value of the measured operating current can be related to one another and used as spark parameter FP. Alternatively, in case of an impressed operating current IB, the operating voltage UB can be calculated on one hand and can be measured on the other hand and analog to the above described proceeding the spark parameter FP can be determined.

The ratio of a peak value to a root mean square value can be used as crest factor for the operating voltage UB or the operating current IB. If the alternating parameter of the operating voltage UB or the operating current IB is sinusoidal, the peak value corresponds to the amplitude of the alternating parameter.

The generator 23 can be controlled or adjusted. The evaluation unit 30 is configured to evaluate the measured and/or determined operating parameter OP. Depending on the evaluation result, the evaluation unit 30 can create a control signal CS by means of which the operation of generator 23 can be modified or adapted, particularly a characteristic of the electrical operating voltage UB or the electrical operating current IB can be adjusted or modified as explained in more detail in the following.

The supply apparatus 11 is configured to be used in different operating types for different applications, particularly for different treatments of tissue 15. These operating types are denoted as modes MD. An operating person, for example a surgeon, can select or adjust a suitable mode MD for the planned use of the electrosurgical system 10. In the embodiment the supply apparatus 11 provides multiple selectable modes MD, which can be selected, for example, by means of an operating interface 31 of supply apparatus 11.

The mode MD adjusted or selected respectively is characterized by an assigned mode parameter set SP. In the embodiment each mode parameter set SP consists of multiple mode parameters PA that in turn define the operation of generator 23 and thereby particularly preset characteristics of the amount and/or the temporal progress of the impressed operating voltage UB or the impressed operating current IB provided to the operating circuit 17. A mode parameter set SP can comprise one or more of the following mode parameters PA:
a maximum value and/or an average value and/or a root mean square value of the operating voltage UB and/or the operating current IB,
an active power and/or an apparent power and/or a reactive power provided to the operating circuit 17,
a crest factor of the operating voltage UB and/or the operating current IB,
a power factor (e.g. active power in relation to the apparent power),
a frequency of the operating voltage UB and/or the operating current IB,
a waveform of the operating voltage UB and/or the operating current IB,
a modulation type of the operating voltage UB and/or the operating current IB.

For example, a high frequency alternating signal for the operating voltage UB or the operating current IB can be selected as modulation type, which can be preset continuously (cw) or quasi-continuously or with interruption (with longer off-periods between subsequent on-periods than in case of a quasi-continuously waveform).

The high frequency alternating signal form has a polarity change in the embodiment and is preferably without direct component. For example, it can have a sinusoidal progress.

Depending on the set mode, the frequency of the high frequency alternating signal can vary and can be in the range of 100 kHz to 4 MHz, whereby it can be preferably minimum 300 kHz.

The direct signal form has, according to the example, a direct component with an absolute value higher than zero and can be particularly configured without polarity change of the signal. The direct signal form is particularly clocked and can be switched between two and particularly exactly two values, particularly a direct signal value unequal to zero and a direct signal value equal to zero.

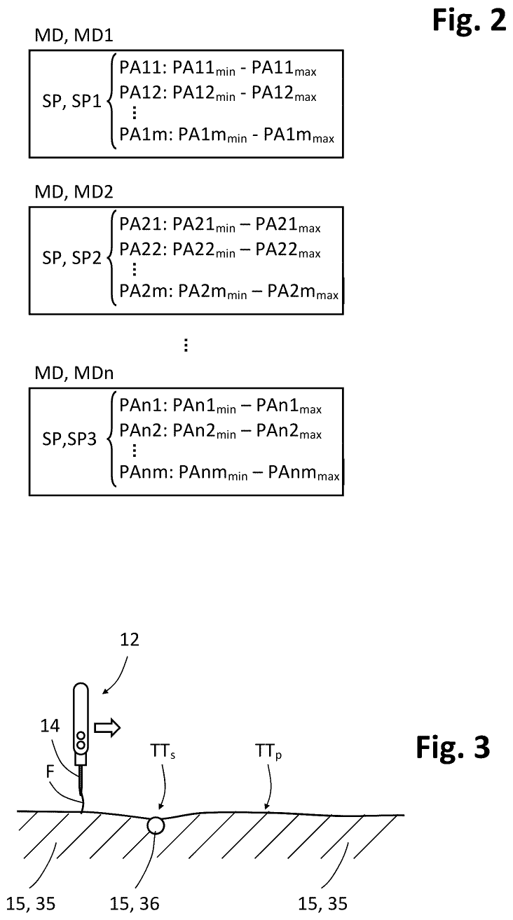

In FIG. 2 multiple available modes MD1 to MDn with the respectively associated mode parameter set SP1 to SPn are schematically indicated. For each mode parameter PA an allowable value range can be defined. With reference to FIG. 2, the value ranges are illustrated by a minimum value and a maximum value respectively. For example, the first mode parameter PA11 of first mode parameter set SP1 has a value range of PA11ₘᵢₙ to PA11ₘₐₓ. Each mode parameter PA can be adjusted or modified within its assigned value range without leaving the respective mode MD.

The treated biological tissue 15 can vary depending on the application or the kind of treatment with regard to the treated tissue type TT. Tissue types TT can be, for example, connective tissue 35, tissue of a blood vessel 36, muscle tissue, neural tissue or epithelial tissue. By way of example, connective tissue 35 and a blood vessel 36 are highly schematically illustrated in FIG. 3. In the shown application example for the tissue types TT of biological tissue 15 to be treated the connective tissue 35 represents a primary tissue type TTₚ and the blood vessel 36 represents a secondary tissue type TTₛ.

Depending on the tissue type TT to be treated (here: primary tissue TTₚ), the operating person of the electrosurgical system 10 or the surgeon can select or adjust an appropriate mode MD. However, applications exist in which the treated tissue 15 does not exclusively correspond to the primary tissue type TTₚ, but is at least locally interfused by tissue structures deviating therefrom. The currently used mode MD can thus be not optimally suitable for this deviating tissue type (here: secondary tissue type TTₛ). Only by way of example, in FIG. 3 the application during cutting of connective tissue 35 is illustrated, which is interfused with fine blood vessels 36 of very small cross-section. If by means of the electrosurgical instrument 12 connective tissue 35 is treated, for example cut, thereby also one or more smaller blood vessels 36 are separated. The mode MD selected for cutting of connective tissue 35 is, however, not optimally suitable for separation of the blood vessels 36. It is therefore provided to at least temporarily adapt one or more of the mode parameters PA of the selected mode MD if a treatment of a secondary tissue type TTₛ (e.g. blood vessel 36) deviating from the primary tissue type TTₚ (e.g. connective tissue 35) is determined. This adaption is carried out automatically without the requirement to manually leave or adapt the mode.

The adaption of the at least one mode parameter PA is, for example, so that the respective mode parameter can be switched between a primary parameter value (or primary condition) and a secondary parameter value (or secondary condition) if the tissue type TT changes from the primary tissue type TTₚ to the secondary tissue type TTₛ or vice versa. Depending on whether the primary tissue type TTₚ or the secondary tissue type TTₛ is treated by means of the electrosurgical instrument 12, the respective concerned mode parameter PA is either set to the primary parameter value (or primary condition) for treatment of the primary tissue type TTₚ or to the secondary parameter value (or secondary condition) for treatment of the secondary tissue type TTₛ.

As an option to the embodiment illustrated here, it is also possible to distinguish more than two different tissue types and to switch between three or more tissue types within a set mode MD and to switch the configuration of the mode parameter set SP accordingly between three or more combinations of mode parameters PA. Thereby one combination of mode parameter values is assigned to each tissue type TT. Which of the mode parameters is modified or switched for treatment of different tissue types TT and the number of modified mode parameters depends on the tissue type TT to be treated respectively, i.e. in the embodiment on the primary tissue type TTₚ and the secondary tissue type TTₛ. Therefore, it can be sufficient to modify only one mode parameter PA of the respective mode parameter set SP-however it can also be necessary to modify multiple of the mode parameters PA or all of the mode parameters PA and to adapt them to one another.

For the adaption it is necessary to distinguish the different tissue types TT from one another. For this reason, the evaluation unit 30 is configured to recognize the tissue type TT currently treated by the electrosurgical instrument 12 based on one operating parameter OP or a combination of multiple operating parameters OP.

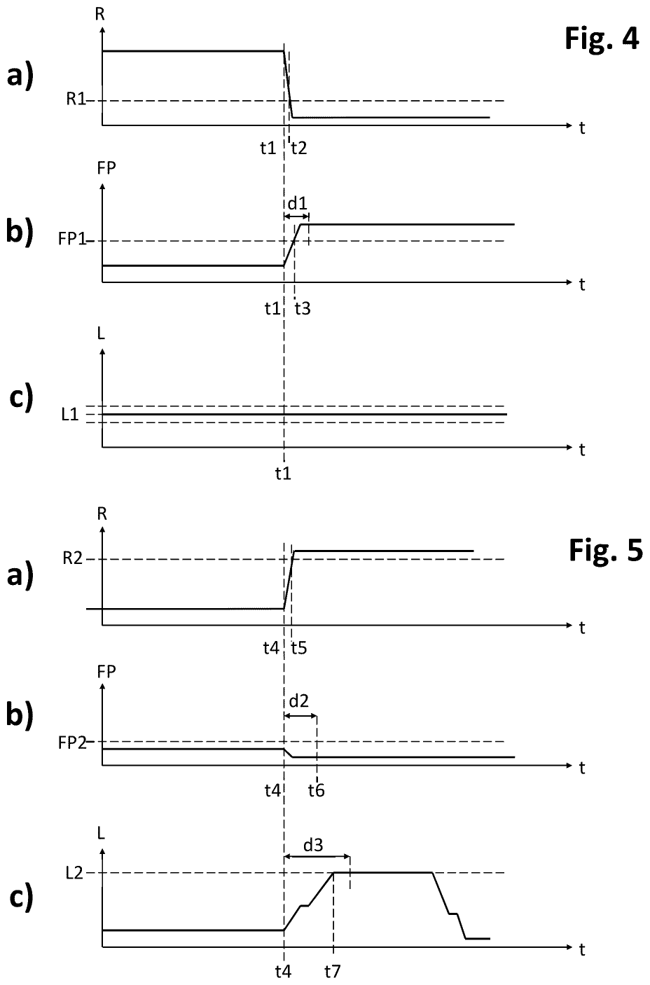

Based on FIGS. 4 to 6, a tissue type recognition is explained exemplarily for the application illustrated in FIG. 3 in which the primary tissue type TTₚ is connective tissue 35 and the secondary tissue type TTₛ is a blood vessel 36.

As illustrated in FIGS. 4 and 5, for recognition of the tissue type TT a combination of multiple operating parameters OP is used, namely the ohmic resistance R of the operating circuit 17 (FIGS. 4a and 5a), the spark parameter FP (FIGS. 4b and 5b) as well as the power factor L (FIGS. 4c and 5c).

In the embodiment treatment of a blood vessel 36 is determined, if the following situation occurs:
(a) The ohmic resistance R is below a first resistance threshold R1. The first resistance threshold R1 can be 500 Ohm, for example.
(b) The spark parameter FP is larger than a predefined first spark parameter threshold FP1. As an option, additionally or alternatively a gradient—for example the average gradient—of the change of the spark parameter FP can be considered, which, for example, must have a minimum absolute value. For example, for this purpose, a change duration d1 can be defined within which the spark parameter FP has to be increased starting from an initial value about a minimum value and/or has to exceed the preset first spark parameter threshold FP1.
(c) The power factor L is substantially constant and remains within a predefined tolerance range, at least after the first spark parameter threshold FP1 has been exceeded and after the first resistance threshold R1 has been underrun. Thereby the power factor L can have a first power factor value L1 of approximately 0.2 (or 20%), for example, and can vary therefrom only within the defined tolerance range.

In FIGS. 4a to 4c the situation of determination of the blood vessel 36 is highly schematically illustrated by way of example. At a first point in time t1 the ohmic resistance R starts to decrease and drops below the first resistance threshold R1 at a second point in time t2. At or after the first point in time t1 the spark parameter FP starts to increase and exceeds the first spark parameter threshold FP1 at a third point in time t3. In the embodiment the power factor L remains within a tolerance range illustrated in dashed lines in FIG. 4c and is illustrated in a simplified manner as a constant having the first power factor value L1. In this example at the third point in time t3 or at least following the third point in time t3, the evaluation unit 30 can determine that the tissue type TT has changed and a blood vessel 36 is treated and, according to the example, cut by means of the electrosurgical instrument 12.

The treatment and, for example, cutting of blood vessels 36 is determined, if the following situation occurs:
(a) The ohmic resistance R is higher than a second resistance threshold R2, for example 1500 Ohm.
(b) The spark parameter FP remains below a second spark parameter threshold FP2 that can be smaller than the first spark parameter threshold FP1—at least during a first minimum duration d2, for example 50 ms.
(c) The power factor L changes excessively and thus has an absolute value change within a predefined change duration d3 exceeding a minimum absolute value. The power factor L can change during treatment of connective tissue over its entire value range up to 100% and can thereby comprise large steps.

The determination of connective tissue 35 is illustrated in FIG. 5 by way of example. At a fourth point in time t4 the ohmic resistance R increases and exceeds the second resistance threshold R2 at a fifth point in time t5. The spark parameter FP is at or after the known resistance change continuously below the second spark parameter threshold FP2 during the minimum duration d2 between the fourth point in time t4 and a sixth point in time t6. After the fourth point in time t4 the power factor L has large variations and steps and changes, by way of example, remarkably between the fourth point in time t4 and a seventh point in time t7 within a change duration d3 (excessive increase). At the seventh point in time t7 or after the seventh point in time t7 it can thus be determined that connective tissue 35 is treated by means of the electrosurgical instrument 12.

Based on the above-explained recognition of the tissue type TT, the transition between connective tissue 35 and blood vessel 36 can be determined accordingly, which is treated by means of the electrosurgical instrument 12. As an example, it is assumed that connective tissue 35 shall be treated with the set mode MD. In addition, it is assumed that at a transition point in time tx the transition from connective tissue 35 (primary tissue type TTₚ) to the tissue of a blood vessel 36 (secondary tissue type TTₛ) is determined and thereupon a modification from one end according to the example multiple mode parameters PA is automatically initiated by means of the evaluation unit 30. The adaption is carried out by control of generator 23 by means of control signals CS.

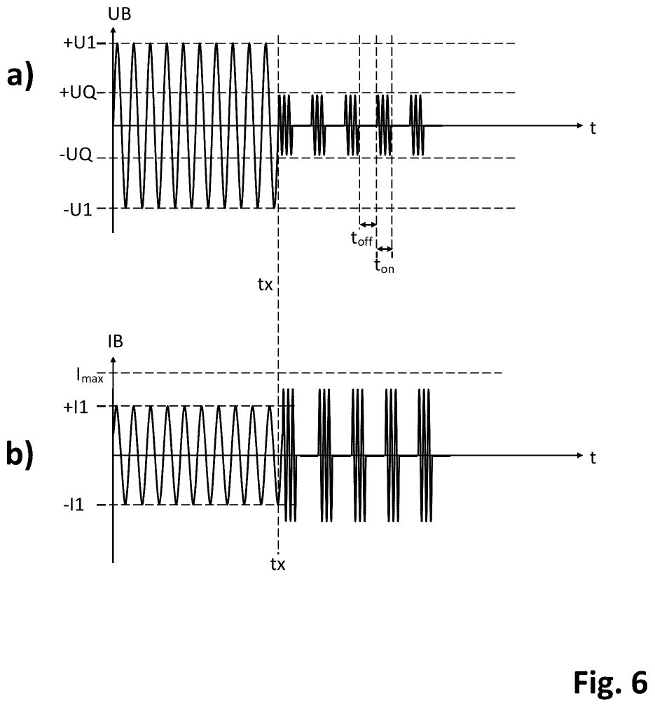

The automatic adaption of the at least one mode parameter PA is by way of example schematically illustrated in FIG. 6. Prior to the transition point in time tx, connective tissue 35 is treated in the embodiment. At the transition point in time tx the transition to a blood vessel 36 is determined.

It is assumed that an operating voltage UB is preset by generator 23. For treatment of connective tissue 35, the operating voltage UB is preset as high frequency alternating signal having an amplitude U1. The operating voltage UB is sinusoidal and, according to the example, free of direct components. It has a frequency in the range of at least 100 kHz or at least 300 kHz up to 4 MHz (FIG. 6a).

It is apparent from FIG. 6b that depending on the sinusoidal operating voltage UB during treatment of connective tissue (primary tissue type TTₚ), a sinusoidal operating current IB having an amplitude I1 results that comprises a phase shift compared to the operating voltage UB. The phase shift can vary excessively during treatment of connective tissue 35 and is illustrated constantly in FIG. 6 only for sake of simplicity.

If at the transition point in time tx the transition of connective tissue 35 (primary tissue type TTₚ) to the blood vessel 36 (secondary tissue type TTₛ) is determined, the following mode parameters PA of mode parameter set SP are changed in the embodiment: the type of modulation or waveform of the preset operating voltage UB, the allowed maximum value of the operating voltage UB, the frequency or period of the operating voltage UB as well as the maximum value of the operating current IB.

At the transition point in time tx the modulation type is changed from a high frequency alternating signal form to a quasi-continuous waveform. In the embodiment during the quasi-continuous waveform the high frequency operating voltage UB is switched on during a pulse duration tₒₙ and switched off during a pause duration t_{off}. The amplitude UQ of the HF alternating voltage is preset to be smaller than the amplitude U1 of the HF alternating voltage before the transition point in time tx. The pulse duration tₒₙ or on-duration and the pause duration t_{off} or off-duration are in this embodiment of equal length, particularly within a single digit millisecond range, for example 5 ms. A frequency with which the high frequency alternating signal is switched on and off during quasi-continuous waveform is therefore 100 Hz, according to the example, and is independent from the specific embodiment preferably not less than 50 Hz.

The operating current IB is clocked adapted to the clocked operating voltage UB and can have a constant phase shift relative to the operating voltage UB.

The illustrations in FIGS. 4 to 6 are highly schematically and have to be understood only qualitatively exemplarily. The temporal progresses of the signals can vary depending on the application.

The recognition of the tissue type TT in the evaluation unit 30 based on the electrical operating parameters OP of operating circuit 17 by evaluation of the absolute values and/or the temporal change of one or more of the operating parameters OP can be carried out very quickly, so to speak in real time. Therefore, a change of the tissue type TT can be determined very simply and sufficiently quick in order to adapt at least one mode parameter PA of the mode parameter set SP of the set mode MD. For example, for distinction of different tissue types TT, only maximum three operating parameters OP have to be determined and evaluated, particularly the ohmic resistance R of the operating circuit 17, the spark parameter FP of spark F as well as the power factor L of the operating circuit 17. The consideration of additional or other operating parameters OP is possible, however, not necessary.

The invention relates to an electrosurgical system 10 as well as a method for the operation thereof. The electrosurgical system 10 comprises a supply apparatus 11 as well as an electrosurgical instrument 12 connected thereto having at least one instrument electrode 14. By means of the instrument electrode 14 biological tissue 15 of a patient can be treated, for example by creation of a spark F between the instrument electrode 14 and tissue 15. The supply apparatus 11 evaluates at least one operating parameter OP of the operating circuit 17 comprising the electrosurgical instrument 12 and the treated tissue 15 and which is supplied with electrical power by supply apparatus 11. A tissue type TT of the treated tissue 15 is recognized by evaluation of at least one operating parameter OP. Thereupon at least one electrical parameter of the provided electrical power can be adapted or modified so that the electrical power provided to the electrosurgical instrument 12 is always optimized for the tissue type TT of the currently treated tissue 15.

### LIST OF REFERENCE SIGNS

10 electrosurgical system
11 supply apparatus
12 electrosurgical instrument
13 cable
14 instrument electrode
15 biological tissue
16 optical detection device
17 operating circuit
18 neutral electrode
19 electrical line
23 generator
24 voltage source
25 current source
26 output of supply apparatus
30 evaluation unit
31 operating interface
35 connective tissue
36 blood vessel
CS control signal
d1 change duration
d2 minimum duration
d3 change duration
F spark
FP spark parameter
FP1 first spark parameter threshold
FP2 second spark parameter threshold
I1 amplitude of operating current
operating current
Imax maximum value of operating current
L power factor
L1 first power factor value
L2 second power factor value
MD mode
OP operating parameter
PA mode parameter
R ohmic resistance of operating circuit
R1 first resistance threshold
R2 second resistance threshold
SP mode parameter set
t time
t1 first point in time
t2 second point in time
t3 third point in time
t4 fourth point in time
t5 fifth point in time
t6 sixth point in time
t7 seventh point in time
t_{off} pause duration
tₒₙ pulse duration
tx transition point in time
TT tissue type
TTₚ primary tissue type
TTₛ secondary tissue type
U1 amplitude of operating voltage
UB operating voltage
UQ amplitude of operating voltage during quasi-stationary waveform

1. An electrosurgical system (10), comprising:
an electrosurgical instrument (12) having at least one instrument electrode (14) for treatment of biological tissue (15); and
a supply apparatus (11) to which the electrosurgical instrument (12) is connected, so that a closed operating circuit (17) can be formed at least through the electrosurgical instrument (12) and a treated tissue (15);
wherein the supply apparatus (11) is configured to provide the electrosurgical instrument (12) with an electrical operating voltage (UB) and/or an electrical operating current (IB), wherein at least one mode (MD) can be set and individual ones of the at least one mode (MD) are defined by one mode parameter set (SP) of multiple mode parameters (PA);
wherein the supply apparatus (11) is additionally configured to detect at least one electrical operating parameter (OP) of the closed operating circuit (17); and
wherein the supply apparatus (11) is additionally configured to determine, based on the at least one electrical operating parameter (OP), whether a treated tissue type (TT) treated by the electrosurgical instrument (12) changes from a primary tissue type (TTₚ) assigned to a set mode (MD) of the at least one mode (MD) to a secondary tissue type (TTₛ) different from the primary tissue type (TTₚ), and in case of a determined change, the supply apparatus (11) is configured to change at least one of the multiple mode parameters (PA) of the mode parameter set (SP) within the set mode (MD) for adaption to the secondary tissue type (TTₛ).

2. The electrosurgical system according to claim 1, wherein individual ones of the multiple mode parameters (PA) of a respective mode parameter set (SP) comprise a defined value range within which a value of the individual ones of the multiple mode parameters (PA) is adapted.

3. The electrosurgical system according to claim 1, wherein the supply apparatus (11) is configured to change the at least one mode parameter (PA) back to a value that was initially set prior to the adaption to the secondary tissue type (TTs) if the supply apparatus (11) determines, based on the at least one electrical operating parameter (OP), that the tissue type (TT) has changed from the secondary tissue type (TTₛ) back to the primary tissue type (TTₚ).

4. The electrosurgical system according to claim 1, wherein the supply apparatus (11) is configured to change the at least one mode parameter (PA) back to a value that was initially set prior to the adaption to the secondary tissue type (TTₛ) if a termination condition has been fulfilled.

5. The electrosurgical system according to claim 4, wherein the termination condition is fulfilled if a predefined maximum duration of time has expired since a change of one or more of the multiple mode parameters (PA) from a respective primary parameter value (Vₚ) to a respective secondary parameter value (Vₛ).

6. The electrosurgical system according to any of the preceding claim 1, wherein the supply apparatus (11) is configured to distinguish connective tissue (35) as a primary tissue type (TTₚ) and a blood vessel (36) as a secondary tissue type (TTₛ) from one another.

7. The electrosurgical system according to claim 1, wherein the supply apparatus (11) is configured to change one or more of the following mode parameters (PA), if a change of the treated tissue type (TT) has been determined:
a maximum value and/or average value and/or root mean square value of the electrical operating voltage (UB);
a maximum value and/or average value and/or root mean square value of the electrical operating current (IB);
a crest factor of the electrical operating voltage (UB) and/or the electrical operating current (IB);
an active power and/or apparent power and/or reactive power of the electrical operating current (IB);
a power factor;
a frequency and/or waveform of the electrical operating voltage (UB);
a frequency and/or waveform of the electrical operating current (IB); and
a modulation type of the electrical operating voltage (UB) and/or the electrical operating current (IB).

8. The electrosurgical system according to claim 7, wherein the supply apparatus (11) is configured to change the mode parameters (PA) as follows if the treated tissue type (TT) treated by the electrosurgical instrument (12) changes from connective tissue (35) as primary tissue type (TTₚ) to a blood vessel (36) as secondary tissue type (TTₛ):
an allowed maximum absolute value of the electrical operating voltage (UB) is reduced;
an allowed maximum absolute value of the electrical operating current (IB) is increased; and
a modulation type is switched from a high frequency alternating signal form for the electrical operating voltage (UB) and the electrical operating current (IB) to a quasi-continuous waveform.

9. The electrosurgical system according to claim 1, wherein the supply apparatus (11) is configured to detect the electrical operating voltage (UB) and the electrical operating current (IB) and to evaluate one or more of the following operating parameters (OP) for recognition of a change of the treated tissue type (TT):
an ohmic resistance and/or impedance of the closed operating circuit (17);
a spark parameter indicating an influence of a spark between the at least one instrument electrode (14) of the electrosurgical instrument (12) and the treated tissue (15) on the electrical operating current (IB) and/or the electrical operating voltage (UB);
a maximum value and/or an average value and/or a root mean square value and/or a crest factor of the electrical operating voltage (UB);
a maximum value and/or an average value and/or a root mean square value and/or a crest factor of the electrical operating current (IB); and
an active power and/or a reactive power and/or an apparent power and/or a power factor.

10. The electrosurgical system according to claim 9, wherein the supply apparatus is configured to detect a change from the primary tissue type (TTₚ) of a connective tissue (35) to the secondary tissue type (TTₛ) of a blood vessel (36) in response to:
(i) the ohmic resistance (R) decreasing;
(ii) the spark parameter (FP) increasing by a minimum value within a predefined change duration of time (d1) or exceeding a predefined spark parameter threshold; and
(iii) the power factor (L) remaining within a predefined tolerance range.

11. The electrosurgical system according to claim 9, wherein the supply apparatus (11) is configured to detect a change from the secondary tissue type (TTₛ) of a blood vessel (36) to the primary tissue type (TTₚ) of a connective tissue (35) in response to:
(i) the ohmic resistance increasing;
(ii) the spark parameter (FP) being below a predefined spark parameter threshold during a minimum duration of time; and
(iii) the power factor (L) changing about a minimum absolute value within a predefined change duration of time (d3).

12. The electrosurgical system according to claim 1, characterized in that wherein the electrosurgical instrument (12) and/or the supply apparatus (11) is configured to indicate a change of a mode parameter (PA) or multiple mode parameters (PA) to an operating person.

13. A method for operating an electrosurgical system (10) comprising an electrosurgical instrument (12) having at least one instrument electrode (14) for treatment of biological tissue (15) and a supply apparatus (11) to which the electrosurgical instrument (12) is connected so that at least through the electrosurgical instrument (12) and the biological tissue (15) a closed operating circuit (17) can be formed, wherein the method comprises the following steps:
setting a mode (MD) that is defined by a mode parameter set (SP) of multiple mode parameters (PA);
providing an electrical operating voltage (UB) and/or an electrical operating current (IB) based on the mode (MD) for the electrosurgical instrument (12) by the supply apparatus (11);
detecting at least one electrical operating parameter (OP) of the operating circuit;
determining, based on the at least one electrical operating parameter (OP), whether a tissue type (TT) treated by the electrosurgical instrument (12) changes from a primary tissue type (TTₚ) assigned to the mode (MD) to a secondary tissue type (TTₛ) different from the primary tissue type (TTₚ); and
changing at least one of the mode parameters (PA) of the mode parameter set (SP) within the mode (MD), in case a change of the tissue type (TT) has been determined, from a primary parameter value (Vₚ) to a value different therefrom.
